(19) United States
(12) Patent Application Publication
(10) Pub. No.: US 2014/0323702 A1
(43) Pub. Date: Oct. 30, 2014

(54) METHOD FOR PRODUCING A PROTEIN HYDROLYSATE

(71) Applicant: OTC GmbH, Oberhausen (DE)

(72) Inventors: Gerd Dahms, Duisburg (DE); Andreas Jung, Duisburg (DE)

(21) Appl. No.: 14/355,453

(22) PCT Filed: Nov. 30, 2012
(86) PCT No.: PCT/EP2012/004940

§ 371 Date: Apr. 30, 2014

(30) Foreign Application Priority Data
Nov. 30, 2011 (DE) 10 2011 055 889.6

(51) Int. Cl. C07K 1/12; C07K 1/34

(52) U.S. Cl. CPC C07K 1/122; C07K 1/34
USPC 530/407

(57) ABSTRACT

The invention relates to a method for producing a protein hydrolysate. In particular, the invention relates to a method for producing a keratin hydrolysate. A method for producing a protein hydrolysate is proposed, comprising: providing a protein source in the form of an aqueous suspension; adding a complexing agent to the provided protein source suspension; adding a base to the provided protein source suspension; heating the obtained mixture to a temperature ≧60° C.; adjusting the pH value of the mixture to a value between ≧pH 2 and ≦pH 8; and filtering the mixture.

### CROSS-REFERENCE TO RELATED APPLICATIONS

This application is a National Stage of International Application No. PCT/EP2012/004940, filed on Nov. 30, 2012, and published in German as WO 2013/079208 A1 on Jun. 6, 2013. This application claims the benefit and priority of German Application No. 10 2011 055 889.6, filed on Nov. 30, 2011. The entire disclosures of the above applications are incorporated herein by reference.

### BACKGROUND

This section provides background information related to the present disclosure which is not necessarily prior art.

1. Technical Field

The present invention relates to a method for producing a protein hydrolysate. Specifically, the present invention relates to a method for producing a keratin hydrolysate.

2. Discussion

Nowadays protein hydrolysates are used in a variety of industries as a raw material due to their manifold applications and their generally good availability. Herein an exemplary field of application to be cited is the manufacture of cosmetic products or body care products. As a source of raw materials for the production of protein hydrolysates vegetable or animal substrates are used. Here, also residual materials can be used, which is generated in the processing of corresponding plants or animals in other areas, such as in the food industry.

A protein hitherto used only to a very small extent in the production of hydrolysates is keratin, although corresponding keratin sources are available in large quantities. Thus, for example, in the field of poultry farming feathers having a very large keratin content are obtained in large quantities. However, while the hydrolysis of proteins of vegetable origin or collagenous proteins of animal origin is usually not a problem, the processing of keratin-containing protein sources into corresponding hydrolysates is significantly more difficult. This difficulty compared to other proteins is caused in particular by the structure of the keratin, which compared to other proteins has a large number of disulfide bridges. Due to these disulfide bridges the keratin achieves its high strength. Simultaneously, however, these disulfide bridges impede the decomposition of the keratin during the hydrolysis.

In order to provide the keratin present, for example, in feathers, wool or horn for a further processing the protein scaffold has to be destroyed. For this purpose, for example, a hydrolytic cleavage in an aqueous solution is known from the prior art. Here, a keratin-containing raw material such as wool is subjected to an elevated temperature (e.g. about 150° C.) and an elevated pressure (e.g. about 350 kPa) for a time period of 30 to 70 minutes. Under these conditions the keratin will be denatured and can then be easily cleaved, however, these reaction conditions lead to an irreversible destruction of parts of the amino acids. This, however, has a sustainable effect on the quality of the hydrolysates.

Furthermore, from the prior art it is known to heat keratin-containing raw materials in strongly acidic or strongly alkaline solution at temperatures of about 100° C. Such a treatment is suitable to break the keratin contained in the raw material, however, even in this case amino acids are irreversibly destroyed.

In order to mitigate these drawbacks it has been attempted to subject keratin-containing materials to an enzymatic cleavage. For example, EP-A-0499261 describes a method for hydrolysing keratin in which a keratin-containing material is at first treated with an aqueous solution containing sulfite ions and is then converted into keratin hydrolysate with the aid of a proteolytic enzyme. The pre-treatment with the solution containing sulfite ions is effected at a pH-value of 6 to 9 at a temperature of 60 to 100° C. over a time period of 10 minutes to 4 hours. The subsequent proteolysis is carried out by multi-stage supply of the pre-treated keratin-containing material into the enzyme-containing hydrolysis mixture. A disadvantage in the method described is the fact that no continuous treatment of keratin-containing material is possible. Moreover, the reaction times required in the enzymatic reaction are very long and a final thermal deactivation of the enzymes used is necessary in which the solutions must be heated to temperatures of about 90° C.

WO 02/36801 A1 discloses a protein hydrolysate which is obtained by continuous enzymatic hydrolysis of a protein-containing substrate, wherein the hydrolysis described is carried out within an extruder.

The methods for hydrolysing keratin-containing substrates or raw materials known from the prior art have a number of drawbacks which up to now impede or even prevent the utilisation of keratin-containing raw materials on a larger scale. The known methods either result in products the use of which in sensitive areas such as cosmetics or body care is not possible because of toxic or potentially toxic ingredients or are not economical due to their long process time or the lack of homogeneity of the resulting hydrolysate.

### SUMMARY OF THE INVENTION

It is therefore an object of the present invention to provide an improved method for producing a protein hydrolysate, in particular on the base of keratin-containing protein sources which allows the provision of a protein hydrolysate in particular with an improved interfacial activity.

This object is achieved by a method according to the teachings of the present disclosure. Embodiments of the method according to the invention can be found in the following description.

Thus, a method for producing a protein hydrolysate is proposed, comprising:

providing a protein source in the form of an aqueous suspension;
adding a complexing agent to the provided protein source suspension;
adding a base to the provided protein source suspension;
heating the resulting mixture to a temperature of ≧60° C.;
adjusting the pH-value of the mixture to a value between ≧pH 2≦pH 8; and filtering the mixture.

Surprisingly it has been found that the thus produced protein hydrolysates exhibit a markedly improved interfacial activity as well as a negative redox potential. Herein improved interfacial activity means in particular that the protein hydrolysates produced according to the present invention lower the interfacial tension in a region hitherto known only from conventional O/W emulsifiers. The protein hydrolysates produced according to the present invention thus offer themselves as emulsifiers/dispersants for respective industrial applications.

Surprisingly it has been found that by adding a complexing agent to the reaction mixture it is possible to prevent or at least to significantly reduce a recombination of the disulfide bridges cleaved by the hydrolysis. Without being bound by theory it is assumed that by adding an appropriate complexing agent the metal ions necessary for the catalytic recombination of the disulfide bridges such as Mn²⁺, Fe²⁺, or Cu²⁺ are no longer available in the reaction solution for a necessary catalysis. By means of this the production of malodorous by-products of the hydrolysis is significantly reduced or even avoided.

According to a preferred embodiment of the method according to the present invention the complexing agent is selected from the group consisting of ethylenediaminetetraacetic acid (EDTA), nitrilotriacetic acid (NTA), ethylene-glycol-bis(aminoethyl ether)-N,N′-tetraacetic acid (EGTA), ethylenediamine disuccinic acid (EDDS), citric acid, 2,3-dihydroxybutanedioic acid, piroctone olamine, phytochelatine, natural or synthetic polypeptides and amino acids, crown ether, derivatives or mixtures thereof. These complexing agents have been found to be particularly suitable to complex the metal ions necessary for the catalytic recombination of the cleaved disulfide bridges under the hydrolysis conditions and thus to reduce or avoid the production of malodorous by-products.

A “protein hydrolysate” in the sense of the invention means a mixture comprising at least about 15 wt. % of polypeptides or oligopeptides which have been produced by chemical cleavage of the protein to be hydrolysed. Herein the polypeptides or oligopeptides predominantly have a molecular weight which is lower than the molecular weight of the protein prior to the hydrolysis.

The protein hydrolysates produced by the method according to the present invention can in principle be produced from any suitable protein sources. Preferably, however, in the method according to the invention protein sources are used which contain at least keratin as a protein. In the context of a preferred embodiment of the method according to the present invention the derived protein hydrolysates thus include hydrolysis products as obtainable by the degradation of keratin. However, this does not preclude that for the production of protein hydrolysates according to the invention, for example, protein sources are used which contain more than one type of protein. Suitable examples are protein sources which in addition to keratin contain collagen or gluten or both.

In a preferred embodiment protein-containing natural products in particular those derived from keratin-containing natural products serve as a protein source for use in the method according to the present invention. As protein-containing natural products, for example, natural materials containing plant proteins such as corn, wheat, barley, soy or materials containing animal protein products such as slaughterhouse waste, wool, feathers, hair, hoofs, horns, bristles and the like as obtained in the processing of carcasses are suited. In addition, maritime protein sources such as fish waste, waste from shellfish and algae can be used as raw materials in the method according to the present invention. Particularly suitable in the context of the present invention are feathers, especially chicken feathers.

In a further embodiment of the method according to the present invention a keratin-containing substrate such as horn, hooves, wool or feathers is used as a protein source in a size reduced form. Suitable size reduction methods, for example, are cutting, shredding or grinding. In particular with the use of feathers as a keratin-containing protein source these are preferably provided in the form of a feather powder. Suitable size reduction stages for the keratin-containing protein source, for example, are sizes of about 2 cm in the longest dimension up to several μm. If feathers are used as the keratin-containing protein source, these may, for example, be such pre-reduced in size that the quills are broken and the feathers have a size of about 1 cm. Preferably, however, a keratin-containing protein source is provided in the form of a powder having an average particle size of about 10 μm to 1 mm.

According to a further embodiment of the method according to the present invention a protein suspension with a solids content between ≧15 wt. % and ≦70 wt. %, preferably between ≧20 wt. % and ≦60 wt. %, more preferably between ≧25 wt. % and ≦40 wt. % is provided. It has been shown that a suspension with such a solids content enables a good processability at a high yield of hydrolysate.

In a further embodiment of the method it may be provided that the suspension provided includes a dispersant. Suitable dispersants are, for example, surfactants such as anionic surfactants, cationic surfactants or nonionic surfactants. Examples of suitable anionic surfactants include alcohol sulfates, alcohol ether sulfates and protein surfactants and/or surfactants based on amino acids. Examples of cationic surfactants are cetyltrimethylammonium bromide (CTAB) and cetyltrimethylammonium chloride (CTAC). Examples of nonionic surfactants are alkoxylates or alkyl polyglucosides. Here, it has been found that the hydrolysis result with respect to the molecular weight distribution of the obtained hydrolysate is independent of the type of the surfactant used. In a particularly preferred embodiment of the method a protein hydrolysate derived from a keratin-containing protein source is used as a surfactant. Thereby the amount of foreign substances within the reaction mixture is reduced. Herein dispersants, for example, serve to increase the reactive surface and to improve the wettability of the protein sources used.

In a preferred embodiment of the method the surfactant may be present in the provided protein source suspension at a concentration between 0.1 wt. % and 50.0 wt. %, preferably between 0.1 wt. % and 20.0 wt. %.

According to a further embodiment of the method a base is added which is selected from the group consisting of alkalihydroxide, alkaline earth hydroxide, calcium oxide, organic bases or mixtures thereof. Herein, it may in particular be provided that the base is selected from a group consisting of NaOH, KOH, Ca(OH)₂ and CaO. It has surprisingly been found that a sufficient hydrolysis of the protein sources is also possible by use of these low-priced and environmentally acceptable bases.

Herein, in one embodiment of the method it may be provided that the base is added in a ratio of between 1:3 and 1:7 based on the solids content in the protein suspension. Herein, the ratio is to be understood such that with respect to one part of the base 3 to 7 parts solids content are added to the protein suspension. Herein the adjusting pH-value is in a range of ≧pH 10, preferably between ≧pH 11 and ≦pH 14.

According to a further embodiment of the method the added complexing agent, for example, selected from the group consisting of ethylenediaminetetraacetic acid (EDTA), nitrilotriacetic acid (NTA), ethylene-glycol-bis(aminoethyl ether)-N,N′-tetraacetic acid (EGTA), ethylenediamine disuccinic acid (EDDS), citric acid, 2,3-dihydroxybutanedioic acid, phytochelatine, natural or synthetic polypeptides and amino acids, crown ether, derivatives or mixtures thereof, may be present in the reaction solution in a concentration between 1*10⁻⁶ wt. % and 10 wt. %, preferably between 1*10⁻⁶ wt. % and 5 wt. %. Such a concentration has been shown to be sufficient to substantially prevent the production of malodorous by-products.

According to a further embodiment of the method it may be provided that a reducing agent is added to the protein source suspension provided. The addition of a reducing agent facilitates the cleavage of the disulfide bridges present in the keratin-containing proteins. Here, according to the invention inorganic and/or organic reducing agents can be used. Suitable inorganic reducing agents are, for example, alkali dithionite, alkali hydrogen sulfite, alkaline earth hydrogen sulfite or mixtures thereof. Here, in particular sodium dithionite has been found to be a preferred reducing agent, since it is cheap and essentially environmentally harmless as a food technologically approved substance. Examples of organic reducing agents are hydrazine, cystine (dimer of cysteine), glutathione disulfide (GSSG) and derivatives or mixtures thereof.

The reducing agent may be added in the method according to the present invention at a concentration between 1:20 and 1:300 with respect to the solids content within the protein suspension. Here, the ratio is to be understood such that 20 parts solids content per one part reducing agent are present in the protein suspension.

According to a further embodiment of the method it may be provided that the resulting mixture of protein suspension, base, complexing agent and optionally a reducing agent is heated under an elevated pressure, preferably between ≧1100 mbar and ≦4000 mbar, more preferably between ≧1500 mbar and ≦3000 mbar. Herein, it may in particular be provided that the resulting mixture is heated to a temperature between 100° C. and 150° C., preferably between 110° C. and 140° C. It has been shown that an increase of the pressure and/or an increase of the temperature lead to a significant reduction of the reaction time required. While at a set temperature of ≧60° C. to ≦100° C. under normal pressure a reaction time of about 4 hours is sufficient to achieve an economically reasonable hydrolysis result, the reaction time with an increase of the pressure and/or the temperature can be reduced to 1.0 to 2.0 hours, preferably 1.5 hours. As a result of this due to the energy saved significant economic and environmental benefits of the process management are obtained.

According to a further embodiment of the method according to the present invention it may be provided that an acid is added for adjusting the pH-value to a value between ≧pH 2 and pH≦8. Preferably the added acid is selected from the group consisting of halogen acids, in particular hydrochloric acid or hydrobromic acid, sulfuric acids, phosphoric acids, carboxylic acids, hydroxycarboxylic acids or mixtures thereof. The term sulfuric acids and phosphoric acids means corresponding oxidation stage variants of sulfur or phosphorus-based acids; halogen acids in this context include oxidation stage variants of the halogen-based acids. By the selection of the acid from the group mentioned above it is advantageously achieved that the total residues resulting from the hydrolysis reaction are environmentally harmless, so that in addition to the desired hydrolysate exclusively biologically harmless and degradable by-products and residues arise.

In a further embodiment of the method a filtration aid is added to the mixture prior to the filtration step. Herein, the temporal specification “prior to the filtration step” means that the filtration aid may be added to the reaction mixture immediately prior to filtration step, in the course of the hydrolysis reaction or even at the beginning. Suitable filtration aids are, for example, those based on Kieselguhr or diatomaceous earth, silicon dioxide or aluminosilicates such as zeolites and activated carbon. Activated carbon may already be added to the reaction solution at the beginning of the method according to the present invention. The filtration step can be accomplished using known filter technologies such as filtration through so-called filter bags with different pore sizes (from 1 μm up to 200 μm) as well as through suction or pressure filtration over filter plates or filter membranes also with different pore sizes. Moreover, centrifuges can be used which are capable of separating the solid components of the reaction mixture from the liquid phase by means of a filter cloth. Here, not only the rotational speed of the centrifuge, but also the porosity of the filter cloth can be varied in order to achieve an effective separation.

According to a further embodiment of the method according to the present invention it can be provided to solidify the hydrolysate and the resulting hydrolysate solution, respectively, in a process step implemented subsequently to the filtration step. Such a solidification may be implemented, for example, by lyophilisation and/or spray drying. The solid hydrolysate thus obtained may be transported in an advantageous manner and can readily be used in a variety of industrial processes due to its water solubility.

It has been found that a keratin hydrolysate produced by the method according to the present invention has a molecular weight distribution between 200 g/mol and 100,000 g/mol, preferably between 4000 g/mol and 5500 g/mol, which substantially corresponds to the molecular weight distribution of keratin hydrolysates known in the prior art which, for example, were obtained by enzymatic reactions.

Hereinafter the method according to the present invention will be explained with reference to examples.

In a heatable stainless steel vessel the mixtures listed in the following table were prepared and hydrolysed under the conditions indicated. To this end, water was placed in the vessel in the respective specified amounts and subsequently the vessel was inerted by repeatedly drawing vacuum and flooding with nitrogen gas. Subsequently EDTA was added and dissolved as a complexing agent in the specified amount. Sodium dithionite was added as a reducing agent in the specified amount to the mixture thus obtained. The respective specified amounts of a feather powder and a base (here, caustic soda) were added to this solution. Then the vessel was closed and heated to the specified temperature for the time also specified in the table. After lapse of the reaction period the vessel was cooled down to 70° C. and flooded with nitrogen. Celite 545 was added as a filtration aid (in each case approximately 34 kg per 500 kg reaction mixture) to the corresponding cooled reaction solution. Subsequently the reaction solution was cooled down to about 47° C. and adjusted to a pH-value between 4.6 to 5.9 by the specified acid before the solution was filtered. The hydrolysate contained in the filtrate after an appropriate drying process exhibited the stated properties in terms of ash content and a mean molecular weight.

TABLE 1
Hydrolysis experiments

| | Experiment No. | | | | | | |
|---|---|---|---|---|---|---|---|
| | 1 | 2 | 3 | 4 | 5 | 6 | 7 |
| | [wt. %] | [wt. %] | [wt. %] | [wt. %] | [wt. %] | [wt. %] | [wt. %] |
| Phase A | | | | | | | |
| Feather powder | 17.8250 | 23.0830 | 23.0681 | 27.0252 | 26.6184 | 0.0000 | 23.5082 |
| Phase B | | | | | | | |
| Water | 71.3000 | 69.2489 | 69.2042 | 67.5630 | 67.4333 | 80.2568 | 68.5656 |
| Plantacare 2000 UP | 4.4562 | 0.0000 | 0.0000 | 0.0000 | 0.0000 | 0.0000 | 0.0000 |
| (Decyl Glucoside) | | | | | | | |
| Lamepon S (Potassium | 0.0000 | 0.4617 | 0.4844 | 0.0000 | 0.0000 | 0.0000 | 0.0000 |
| Cocoyl Hydrolyzed | | | | | | | |
| Collagen) | | | | | | | |
| NaOH (50%) | 6.2387 | 6.9249 | 6.9204 | 5.0672 | 5.8560 | 0.0000 | 7.8361 |
| KOH (50%) | 0.0000 | 0.0000 | 0.0000 | 0.0000 | 0.0000 | 3.4778 | 0.0000 |
| Phase C | | | | | | | |
| EDTA powder | 0.0018 | 0.0046 | 0.0461 | 0.0068 | 0.0035 | 0.2140 | 0.0118 |
| Na hydrosulfite | 0.1782 | 0.2770 | 0.2768 | 0.3378 | 0.0887 | 0.0000 | 0.0784 |
| Total: | 100.0000 | 100.0000 | 100.0000 | 100.0000 | 100.0000 | 100.0000 | 100.0000 |
| Lactic acid 80% | | | X | X | X | X | X |
| Phosphorous acid (H₃P0₃, | | X | | | | | |
| 30%) | | | | | | | |
| Hydrochloric acid (37%) | X | | | | | | |
| Start pH | 12.5 | 13.1 | 12.8 | 12.3 | 11.9 | 13.5 | 12.5 |
| Final pH after adjustment | 5.9 | 4.8 | 4.6 | 5.1 | 4.8 | 5.1 | 4.7 |
| Temperature [° C.] | 85 | 90 | 125 | 90 | 95 | 98 | 130 |
| Reaction time [h] | 4 | 2.5 | 1 | 4 | 4 | 4 | 1.5 |
| Evaporation residue [%] | 13.2 | 12.8 | 19.7 | 18.7 | 18.9 | 22.2 | 25.5 |
| Nitrogen content [%] | 1.46 | 1.5 | 1.97 | 1.65 | 1.84 | 2.61 | 2.28 |
| Molecular weight [Da] | 4800 | 5100 | 5000 | 5100 | 4900 | 5000 | 5100 |

### DETAILED DESCRIPTION OF THE PREFERRED EMBODIMENTS

Example embodiments will now be described more fully with reference to the accompanying drawings.

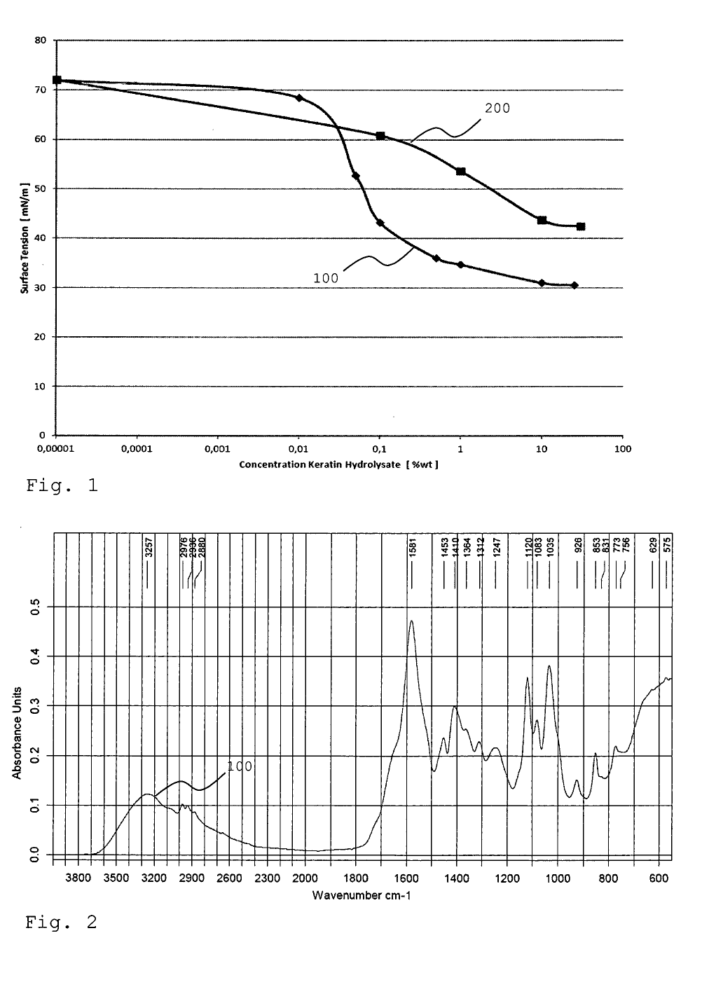

The keratin hydrolysates that can be produced by the method according to the present invention have a critical micelle concentration (CMC) in a range of 0.05 mM to 0.5 mM. Thus, the CMC of the keratin hydrolysate produced by the method according to the present invention is significantly lower than the CMC of keratin hydrolysates known from the prior art. Such a low CMC contributes to an improved effectiveness of the hydrolysates produced according to the present invention as a detergent or surfactant in washing processes. FIG. 1 shows the surface tension of an aqueous solution as a function of the concentration of a keratin hydrolysate 100 produced according to the present invention as well as of a comparative product 100 (Kera-Tein, Tri-K Industr.) at a measurement temperature of 25° C. The critical micelle concentration is obtained from the ordinate value of the inflection point of the respective concentration curve. As can be seen, the keratin hydrolysate produced according to the present invention exhibits a CMC value which compared with comparative product is about two orders of magnitude lower. Thus, taking into account an average molecular weight in the range of the present invention a CMC in the range from about 0.05 mM to 0.5 mM is obtained.

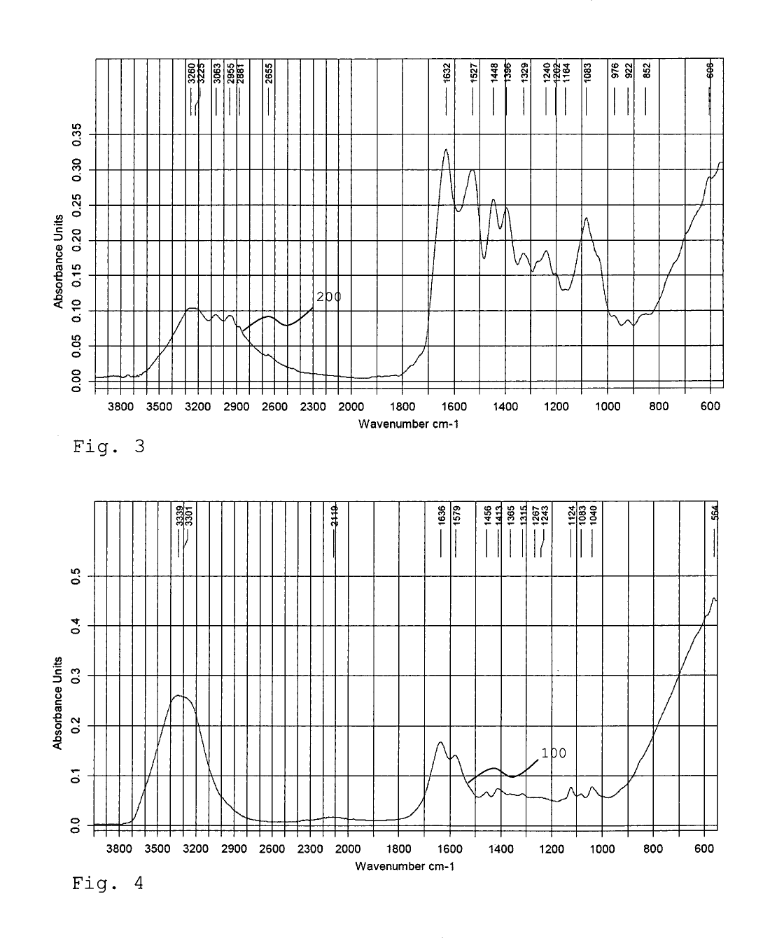

FIG. 2 shows an example of an IR spectrum of a keratin hydrolysate 100 produced according to the present invention as a dry substance. As characteristic bands here in particular the vibrations at 1035 cm⁻¹ and 1120 cm⁻¹ were identified. Here the absorption at 1120 cm⁻¹ is presumably attributable to a NH₂ deformation vibration (rock) of an aliphatic primary amide, whereas the absorption at 1035 cm⁻¹ is attributable to a CO stretching vibration of an aliphatic primary alcohol. FIG. 3 shows the IR spectrum of a comparative product 200 (Kera-Tein, Tri-K Industr.) in which this characteristic absorption bands are absent.

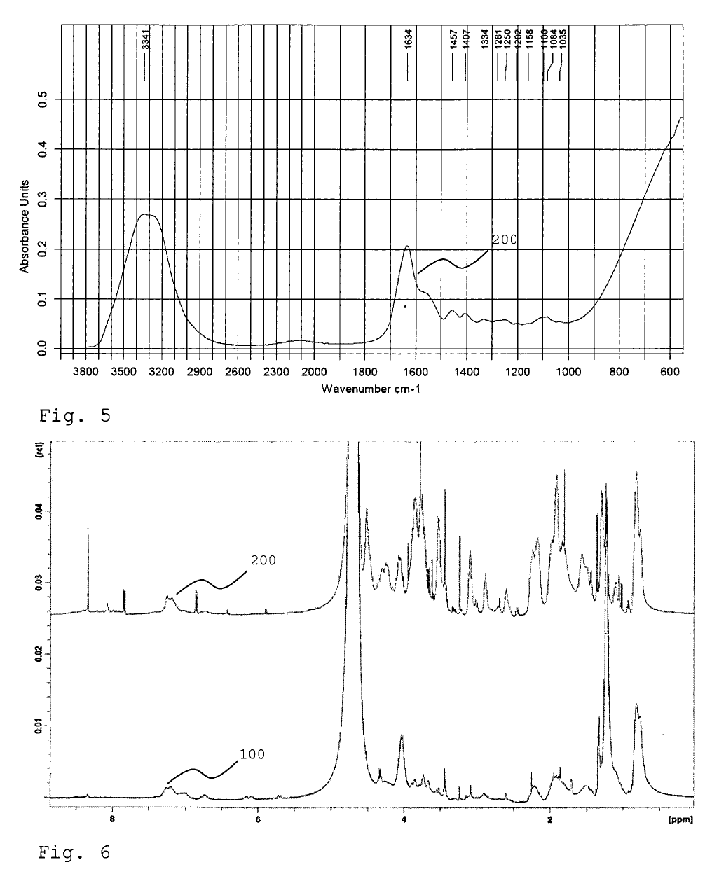

FIG. 4 shows the IR spectrum of a 25 wt. % solution of a keratin hydrolysate 100 in water produced according to the present invention. Within the error range and the expected solution shifts the characteristic bands can also be found here at 1124 cm⁻¹ and 1040 cm⁻¹. In addition, there is a distinct splitting of the band in the amide I region, which could allow conclusions on the secondary structure. The characteristic bands in this region are assigned to a stretching vibration of a carbonyl group, wherein a splitting of this band could be an indication that the excited carbonyl group forms hydrogen bridges to two different binding partners. On the other hand, the amide I region in FIG. 5 which shows the IR spectrum of the comparative product 200 (Kera-Tein) in a solution with identical concentration exhibits no splitting. This leads to the assumption that the keratin hydrolysate produced according to the present invention has a different secondary structure compared to the comparative product.

FIG. 6 shows a comparison between the ¹H-NMR spectra of a keratin hydrolysate 100 produced according to the present invention and the comparative product 200 (Kera-Tein). Both spectra exhibit distinct differences. In particular, the comparative product shows a significantly higher number of signals compared to the hydrolysate produced according to the present invention, which leads to the assumption that the keratin hydrolysate produced by the present invention is present as a significantly more uniform and more defined product.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for producing a protein hydrolysate comprising:
providing a protein source in the form of an aqueous suspension;
adding a complexing agent to the provided protein source suspension;
adding a base to the provided protein source suspension;
heating the resulting mixture to a temperature of ≧60° C.;
adjusting the pH-value of the mixture obtained to a value between ≧pH 2 and ≦pH 8; and
filtering the mixture.

2. The method according to claim 1, wherein a protein source suspension having a solids content between ≧15 wt. % and ≦70 wt. %, preferably between ≧20 wt. % and ≦60 wt. %, more preferably between ≧25 wt. % and ≦40 wt. % is provided.

3. The method according to claim 1, wherein a protein suspension is provided which includes a dispersant, preferably a surfactant.

4. The method according to claim 1, wherein a base selected from the group consisting of alkali hydroxide, alkaline earth hydroxide, calcium oxide or mixtures thereof is added.

5. The method according to claim 1, wherein the base is added in a ratio between 1:3 and 1:7 based on the solids content in the protein suspension obtained.

6. The method according to claim 1, wherein the complexing agent is selected from the group consisting of ethylenediaminetetraacetic acid (EDTA), nitriloacetic acid (NTA), ethylene-glycol-bis(aminoethyl ether)-N,N′-tetraacetic acid (EGTA), ethylenediamine disuccinic acid (EDDS), citric acid, 2,3-dihydroxybutanedioic acid, piroctone olamine, phytochelatine, natural or synthetic polypeptides and amino acids, crown ethers, derivatives or mixtures thereof.

7. The method according to claim 1, wherein a reducing agent is added to the provided protein source suspension.

8. The method according to claim 7, wherein as the reducing agent a compound selected from the group consisting of alkali dithionite, alkali hydrogen sulfite, alkaline earth hydrogen sulfite, hydrazine, cystine (dimer of cysteine), glutathione disulfite (GSSG) or mixtures thereof is added.

9. The method according to claim 7, wherein the reducing agent is added in a ratio between 1:20 and 1:300 based on the solids content in the protein suspension.

10. The method according to claim 1, wherein the resulting mixture is heated at an elevated pressure preferably between ≧1100 mbar and ≦4000 mbar, more preferable between 1500 mbar and 3000 mbar.

11. The method according to claim 10, wherein the mixture is heated to a temperature between 100° C. and 150° C., preferably between 110° C. and 140° C.

12. The method according to claim 1, wherein an acid is added to the mixture in order to adjust the pH-value to a value between ≧pH 2 and pH≦8.

13. The method according to claim 12, wherein the acid is selected from the group consisting of hydrochloric acid, sulfuric acid, phosphoric acid, phosphorous acid, carboxylic acids, hydroxycarboxylic acids or mixtures thereof.

14. The method according to claim 1, wherein a filtration aid is added to the mixture prior to the filtration.

15. The method according to claim 1, wherein the resulting filtrate is subjected to a spray-drying or lyophilisation process.
